(19) United States
(12) Patent Application Publication
(10) Pub. No.: US 2025/0114488 A1
(43) Pub. Date: Apr. 10, 2025

(54) PSMA-TARGETED RADIOPHARMACEUTICALS AND CHECKPOINT INHIBITOR COMBINATION THERAPY

(71) Applicant: Fusion Pharmaceuticals Inc., Hamilton (CA)

(72) Inventors: Natalie Grinshtein, Bothell, WA (US); Shekoufeh Almasi, Oakville (CA); Saleemulla Mahammad, Ancaster (CA)

(21) Appl. No.: 18/833,850

(22) PCT Filed: Jan. 27, 2023
(86) PCT No.: PCT/CA2023/050110

§ 371 Date: Jul. 26, 2024

Related U.S. Application Data
(60) Provisional application No. 63/304,181, filed on Jan. 28, 2022.

(51) Int. Cl. A61K 51/04; A61K 39/00; A61K 51/08; A61P 35/00; C07K 16/28

(52) U.S. Cl. CPC A61K 51/0497; A61K 51/08; A61P 35/00; C07K 16/2818; A61K 2039/507

(57) ABSTRACT

Combination therapies comprising administering PSMA-targeted radiopharmaceuticals and one or more checkpoint inhibitors

[symbol]

### CROSS-REFERENCE TO RELATED APPLICATIONS

This application claims the benefit of U.S. Provisional Patent Application No. 63/304,181, filed on Jan. 28, 2022, the entire content of which is incorporated by reference herein.

### BACKGROUND

Cancer cells employ a variety of mechanisms to escape immune surveillance, including suppression of T cell activation.

The mammalian immune system relies on checkpoint molecules to distinguish normal cells from foreign cells. Checkpoint molecules, expressed on certain immune cells, need to be activated or inactivated to start an immune response. Inhibition of checkpoint proteins results in increased activation of the immune system. Checkpoint inhibition has been explored as a method of immunotherapy for cancer. Upregulation of checkpoint molecules such as programmed death 1 (PD-1), programmed death ligand-1 (PD-L1), and cytotoxic T-lymphocyte-associated antigen 4 (CTLA-4) is naturally meant to restrict the magnitude of the tumor-specific immune response. Thus, blocking these checkpoint molecules results in a more robust and persistent T cell activation. However, checkpoint inhibition can also enable the immune system to attack some normal cells in the body, which can lead to harmful side effects. In addition, only a limited number of cancer types such as melanoma, lung cancer, bladder cancer, and head and neck tumors exhibit intrinsic sensitivity to checkpoint inhibition. Within the tumor types that do respond, the typical overall response rate across patients is only 20-25%.

Therefore, there is a need for improved treatments of cancer. In particular, there is a need for increases in efficacy, which do not enhance toxicity in the patient.

### SUMMARY

The present disclosure encompasses the insight that combining inhibition of checkpoint proteins with a therapy that targets damage to cancer cells may provide a less toxic therapy with improved efficacy. Radioactive decay can cause direct physical damage (such as single or double-stranded DNA breaks) or indirect damage (such as by-stander or crossfire effects) to the biomolecules that constitute a cell. Drugs that deliver radionuclides to cancer cells, i.e., radiopharmaceuticals, provide a mechanism to generate DNA damage with anti-cancer therapeutic effect. The present disclosure provides the combination of ²²⁵Ac-radiopharmaceuticals, specifically, the small molecule-based radiopharmaceuticals targeting Prostate Specific Membrane Antigen (PSMA) positive tumors and using actinium-225 to target cancer cells, with checkpoint inhibitors to treat or ameliorate cancer.

More specifically, provided are methods of treating a mammal having cancers expressing Prostate Specific Membrane Antigen (PSMA), said method comprising:
(i) administering to the mammal an ²²⁵Ac-radiopharmaceutical, wherein the mammal has received or is receiving one or more checkpoint inhibitors;
(ii) administering to the mammal one or more checkpoint inhibitors, wherein the mammal has received or is receiving an ²²⁵Ac-radiopharmaceutical; or
(iii) administering to the mammal one or more checkpoint inhibitors at the same time as administering to the mammal an ²²⁵Ac-radiopharmaceutical,
wherein in each occurrence said ²²⁵Ac-radiopharmaceutical comprises ²²⁵Ac chelated with a compound of Formula I, or a stereoisomer thereof:

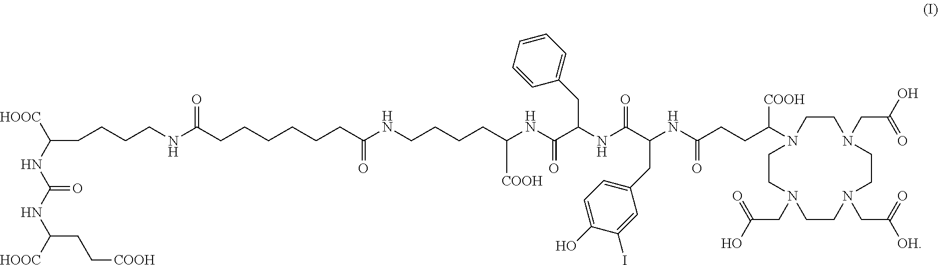 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In some embodiments, said method comprises administering to a mammal one or more checkpoint inhibitors, wherein the mammal has received or is receiving an ²²⁵Ac-radiopharmaceutical.

In some embodiments, said method comprises administering to the mammal an ²²⁵Ac-radiopharmaceutical, wherein the mammal has received or is receiving one or more checkpoint inhibitors.

In some embodiments, said method comprises administering to the mammal one or more checkpoint inhibitors at the same time as administering to the mammal an ²²⁵Ac-radiopharmaceutical.

In some embodiments, the chelator is selected from the group consisting of DOTA, DOTA-GA, NOTA, NODA-GA, and NODA-SA.

In some embodiments, the chelator is selected from the group consisting of DTPA, EDTA, CDTA, DFO, BAT, and HYNIC.

In some embodiments, said ²²⁵Ac-radiopharmaceutical comprises ²²⁵Ac chelated with the following structure:

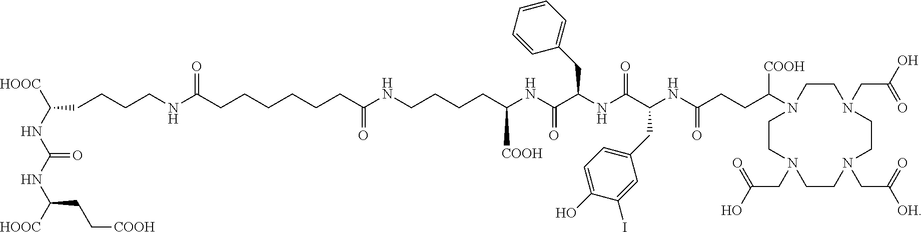 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In some embodiments, the one or more checkpoint inhibitors comprise a PD-1 or PD-L1 inhibitor or a CTLA-4 inhibitor.

In some embodiments, the PD-1 or PD-L1 inhibitor or the CTLA-4 inhibitor is an antibody.

In some embodiments, the one or more checkpoint inhibitors comprise both a PD-1 or PD-L1 inhibitor and a CTLA-4 inhibitor.

In some embodiments, the PD-1 or PD-L1 inhibitor is selected from the group consisting of camrelizumab, cemiplumab, dostarlimab, nivolumab, pembrolizumab, sintilimab, tislelizumab, toripalimab, RMP1-14, atezolizumab, avelumab, and durvalumab.

In some embodiments, the CTLA-4 inhibitor is selected from the group consisting of BMS-986218, BMS-986249, ipilimumab, tremelimumab (formerly ticilimumab, CP-675,206), MK-1308, REGN-4659, and 4F10-11.

In some embodiments, the mammal is a human.

In some embodiments, said ²²⁵Ac-radiopharmaceutical is administered at a dosage of less than 2 MBq/kg of body weight of said mammal.

In some embodiments, said ²²⁵Ac-radiopharmaceutical is administered at a dosage of less than 1 MBq/kg of body weight of said mammal.

In some embodiments, said ²²⁵Ac-radiopharmaceutical is administered at a dosage of less than 750 kBq/kg of body weight of said mammal.

In some embodiments, said ²²⁵Ac-radiopharmaceutical is administered at a dosage of less than 500 kBq/kg of body weight of said mammal.

In some embodiments, said ²²⁵Ac-radiopharmaceutical is administered at a dosage of less than 250 kBq/kg of body weight of said mammal.

In some embodiments, said ²²⁵Ac-radiopharmaceutical is administered at a dosage of less than 100 kBq/kg of body weight of said mammal.

In some embodiments, said ²²⁵Ac-radiopharmaceutical is administered as a unitary dosage of less than 15 MBq to said mammal.

In some embodiments, said ²²⁵Ac-radiopharmaceutical is administered as a unitary dosage of less than 10 MBq to said mammal.

In some embodiments, said ²²⁵Ac-radiopharmaceutical is administered as a unitary dosage of less than 5 MBq to said mammal.

In some embodiments, said checkpoint inhibitor is administered at a dosage of about 1 mg/kg to about 10 mg/kg of body weight of said mammal.

In some embodiments, said checkpoint inhibitor is administered at a dosage of about 5 mg/kg of body weight of said mammal.

In some embodiments, the cancer is selected from the group consisting of prostate cancers, breast cancers, colorectal cancers, renal cell cancers, bladder cancers, testicular-embryonal cancers, neuroendocrine cancers, and brain tumors.

In some embodiments, said cancer is prostate cancer or breast cancer.

In some embodiments, said administration results in a decrease in tumor volume, a stable tumor volume, or a reduced rate of increase in tumor volume. In some embodiments, said administration results in a decreased incidence of recurrence or metastasis.

In some embodiments, said method comprising administering to a mammal one or more checkpoint inhibitors, wherein the mammal has received or is receiving an ²²⁵Ac-radiopharmaceutical comprising ²²⁵Ac chelated with the following structure:

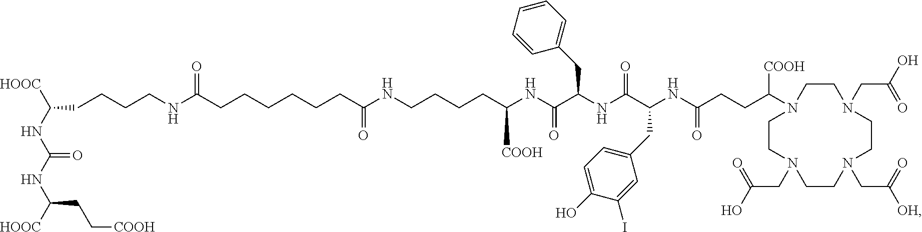 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

wherein the one or more checkpoint inhibitors comprise both a PD-1 inhibitor and a CTLA-4 inhibitor, and wherein said ²²⁵Ac-radiopharmaceutical is administered at a dosage of 0.5-1.5 MBq/kg of body weight of said mammal.

Also provided herein is a use of a compound of Formula I for the manufacture of a medicament for a method of treating a mammal having cancers expressing Prostate Specific Membrane Antigen (PSMA) in a subject in need thereof, said method comprising:
(i) administering to the mammal an ²²⁵Ac-radiopharmaceutical, wherein the mammal has received or is receiving one or more checkpoint inhibitors;
(ii) administering to the mammal one or more checkpoint inhibitors, wherein the mammal has received or is receiving an ²²⁵Ac-radiopharmaceutical; or
(iii) administering to the mammal one or more checkpoint inhibitors at the same time as administering to the mammal an ²²⁵Ac-radiopharmaceutical,
wherein in each occurrence said ²²⁵Ac-radiopharmaceutical comprises ²²⁵Ac chelated
with a compound of Formula I, or a stereoisomer thereof:

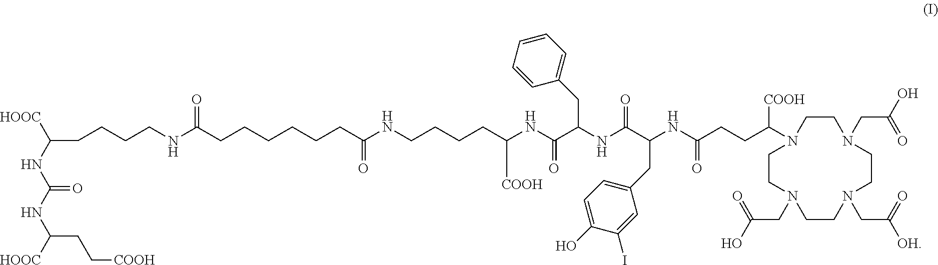 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In another aspect, provided herein is a compound of Formula I for use in treating a mammal having cancers expressing Prostate Specific Membrane Antigen (PSMA) in a subject in need thereof, said method comprising:
(i) administering to the mammal an ²²⁵Ac-radiopharmaceutical, wherein the mammal has received or is receiving one or more checkpoint inhibitors;
(ii) administering to the mammal one or more checkpoint inhibitors, wherein the mammal has received or is receiving an ²²⁵Ac-radiopharmaceutical; or
(iii) administering to the mammal one or more checkpoint inhibitors at the same time as administering to the mammal an ²²⁵Ac-radiopharmaceutical,
wherein in each occurrence said ²²⁵Ac-radiopharmaceutical comprises ²²⁵Ac chelated
with a compound of Formula I, or a stereoisomer thereof:

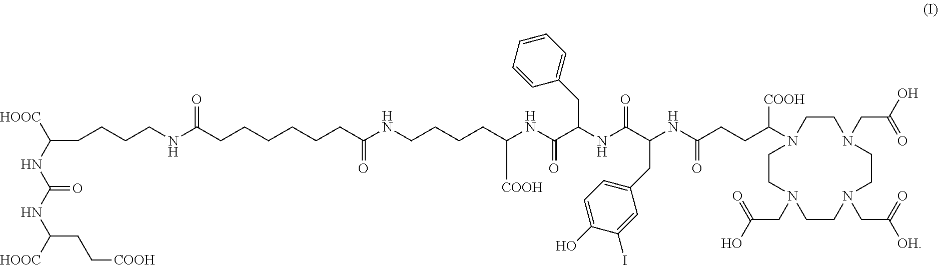 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

### BRIEF DESCRIPTION OF THE DRAWINGS

FIG. 1 illustrates the biodistribution of radiopharmaceutical [¹⁷⁷Lu]-Compound A in a CT-26 syngeneic model.

FIG. 2 illustrates the in vivo efficacy of radiopharmaceutical [²²⁵Ac]-Compound A at different dosages in a CT-26-mFOLH1 syngeneic immunocompetent mouse model.

FIG. 3A and FIG. 3B illustrate increased therapeutic efficacy from the combination of radiopharmaceutical [²²⁵Ac]-Compound A and α-CTLA-4/PD-1 in a CT-26-mFOLH1 syngeneic mouse model.

FIG. 4A and FIG. 4B illustrate improvement of the overall survival in [²²⁵Ac]-Compound A treated mice.

### DETAILED DESCRIPTION

The present disclosure relates to combination therapies for treating cancer using certain radiopharmaceuticals and checkpoint inhibitors in combination. In particular, the radiopharmaceuticals are ²²⁵Ac-chelated small molecules targeting Prostate Specific Membrane Antigen (PSMA).

Radio-labelled targeting moieties (also known as radiopharmaceuticals) are designed to target a protein or receptor (e.g., PSMA) that is upregulated in a disease state and/or specific to diseased cells (e.g., tumor cells) to deliver a radioactive payload to damage and kill cells of interest.

### Definitions

### Chemical Terms

The term “isomer,” as used herein, means any tautomer, stereoisomer, enantiomer, or diastereomer of any compound. It is recognized that the compound of Formula I has one or more chiral centers and, therefore, can exist as stereoisomers, such as diastereomers (e.g., enantiomers (i.e., (+) or (−))). Unless otherwise noted, chemical structures depicted herein encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds may exist in different tautomeric forms, all of the latter being included within the scope of the present disclosure.

The term “diastereomer,” as used herein means stereoisomers that are not mirror images of one another and are non-superimposable on one another.

The term “enantiomer,” as used herein, means each individual optically active form of a compound, having an optical purity or enantiomeric excess (as determined by methods standard in the art) of at least 80% (i.e., at least 90% of one enantiomer and at most 10% of the other enantiomer), preferably at least 90% and more preferably at least 98%.

### Other Terms

As used herein, the term “about” or “approximately” refers to a ±10% variation from the recited quantitative value (and includes the recited quantitative value itself) unless otherwise indicated or inferred from the context. For example, unless otherwise stated or inferred from the context, a dose of about 100 kBq/kg indicates a dose range of 100±10% kBq/kg, i.e., from 90 kBq/kg to 110 kBq/kg, inclusive.

As used herein, the term “administered in combination,” “combined administration,” or “co-administered” means that two or more agents are administered to a subject at the same time or within an interval such that there may be an overlap of an effect of each agent on the patient. Thus, two or more agents that are administered in combination need not be administered together. In some embodiments, they are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s), within 24 hours (e.g., 12, 6, 5, 4, 3, 2, or 1 hour(s), or within about 60, 30, 15, 10, 5, or 1 minute of one another. In some embodiments, the administrations of the agents are spaced sufficiently closely together such that a combinatorial effect is achieved.

As used herein, “administering” an agent to a subject includes contacting cells of said subject with the agent.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “checkpoint inhibitor,” also known as “immune checkpoint inhibitor” or “ICI,” refers to an agent which blocks the action of an immune checkpoint protein, e.g., blocks such immune checkpoint proteins from binding to their partner proteins.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a therapeutic moiety or a targeting moiety.

As used herein, the term “compound,” is meant to include all stereoisomers, geometric isomers, and tautomers of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. C is and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H -and 3H-imidazole, 1H-, 2H- and 4H- 1,2,4-triazole, 1H- and 2H-isoindole, and 1H- and 2H-pyrazole. Tautomeric forms can be in equilibrium or sterically locked into one form by appropriate substitution.

At various places in the present specification, substituents of compounds of the present disclosure are disclosed in groups or in ranges. It is specifically intended that the present disclosure include each and every individual sub-combination of the members of such groups and ranges. For example, the term “C₁₋₆ alkyl” is specifically intended to individually disclose methyl, ethyl, C₃ alkyl, C₄ alkyl, C₅ alkyl, and Co alkyl. Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

As used herein, the terms “decrease,” “decreased,” “increase,” “increased,” or “reduction,” “reduced,” (e.g., in reference to therapeutic outcomes or effects) have meanings relative to a reference level. In some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment.

The term an “effective amount” of an agent (e.g., any of the foregoing conjugates), as used herein, is that amount sufficient to effect beneficial or desired results, such as clinical results, and, as such, an “effective amount” depends upon the context in which it is being applied.

The term “lower effective dose,” when used as a term in conjunction with an agent (e.g., a therapeutic agent) refers to a dosage of the agent which is effective therapeutically in the combination therapies of the invention and which is lower than the dose which has been determined to be effective therapeutically when the agent is used as a monotherapy in reference experiments or by virtue of other therapeutic guidance.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

Compounds may have ionizable groups so as to be capable of preparation as pharmaceutically acceptable salts. These salts may be acid addition salts involving inorganic or organic acids or the salts may, in the case of acidic forms of compounds, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “radiopharmaceutical” or “radioconjugate,” as used herein, refers to any compound or conjugate that includes a radioisotope or radionuclide, such as any of the radioisotopes or radionuclides described herein.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents. In some embodiments, the radionuclide is an alpha-emitting radionuclide. Exemplary radionuclides that may be used in the present disclosure include, but are not limited to, ⁶⁴Cu, ⁶⁷Cu, ⁶⁸Ga, ⁹⁰Y, ¹⁴⁹Tb, ¹⁵³Sm, ¹⁷⁷Lu, ²¹¹At, ²¹²Bi, ²¹²Pb, ²¹³Bi, ²²³Ra, ²²⁵Ac, and ²²⁷Th.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. In the context of cancer treatment, “ameliorating” may include, for example, reducing incidence of metastases, reducing tumor volume, reducing tumor vascularization and/or reducing the rate of tumor growth. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### Checkpoint Inhibitors

In some embodiments, a checkpoint inhibitor is co-administered with a radiopharmaceutical. Generally, suitable checkpoint inhibitors inhibit an immune suppressive checkpoint protein. In some embodiments, the checkpoint inhibitor inhibits a protein selected from the group consisting of cytotoxic T-lymphocyte-associated antigen 4 (CTLA-4), programmed death 1 (PD-1), programmed death ligand-1 (PD-L1), LAG-3, T cell immunoglobulin mucin 3 (TIM-3), T cell immunoreceptor with Ig and ITIM domains (TIGIT), and killer immunoglobulin-like receptors (KIRs).

In some embodiments, the checkpoint inhibitor is capable of binding to CTLA-4, PD-1, or PD-L1. In some embodiments, the checkpoint inhibitor interferes with the interaction (e.g., interferes with binding) between PD-1 and PD-L1.

In some embodiments, the checkpoint inhibitor is a small molecule.

In some embodiments, the checkpoint inhibitor is an antibody or antigen-binding fragment thereof, e.g., a monclonal antibody. In some embodiments, the checkpoint inhibitor is a human or humanized antibody or antigen-binding fragment thereof. In some embodiments, the checkpoint inhibitor is a mouse antibody or antigen-binding fragment thereof.

In some embodiments, the checkpoint inhibitor is a CTLA-4 antibody. Non-limiting examples of CTLA-4 antibodies include BMS-986218, BMS-986249, ipilimumab, tremelimumab (formerly ticilimumab, CP-675,206), MK-1308, and REGN-4659. An additional example of a CTLA-4 antibody is 4F10-11, a mouse monoclonal antibody.

In some embodiments, the checkpoint inhibitor is a PD-1 antibody. Non-limiting examples of PD-1 antibodies include camrelizumab, cemiplumab, dostarlimab, nivolumab, pembrolizumab, sintilimab, tislelizumab and toripalimab. An additional example of a PD-1 antibody is RMP1-14, a mouse monoclonal antibody.

In some embodiments, the checkpoint inhibitor is a PD-L1 antibody. Non-limiting examples of PD-L1 antibodies include atezolizumab, avelumab, and durvalumab.

In some embodiments, a combination of more than one checkpoint inhibitor is used. For example, in some embodiments, both a CTLA-4 inhibitor and a PD-1 or PD-L1 inhibitor is used.

### Subjects

In some disclosed methods, a therapy (e.g., comprising a therapeutic agent) is administered to a subject. In some embodiments, the subject is a mammal, e.g., a human.

In some embodiments, the subject has received or is receiving another therapy. For example, in some embodiments, the subject has received or is receiving a radiopharmaceutical. In some embodiments, the subject has received or is receiving a checkpoint inhibitor.

In some embodiments, the subject has cancer or is at risk of developing cancer. For example, the subject may have been diagnosed with cancer. The cancer may be a primary cancer or a metastatic cancer. Subjects may have any stage of cancer, e.g., stage I, stage II, stage III, or stage IV with or without lymph node involvement and with or without metastases. Provided compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumor.

In some embodiments, the solid tumor cancer is breast cancer, non-small cell lung cancer, small cell lung cancer, pancreatic cancer, head and neck cancer, prostate cancer, colorectal cancer, sarcoma, adrenocortical carcinoma, neuroendocrine cancer, Ewing's Sarcoma, multiple myeloma, or acute myeloid leukemia.

In some embodiments, the cancer is a non-solid (e.g., liquid (e.g., hematologic)) cancer.

### Administration and Dosage

### Effective and Lower Effective Doses

The present disclosure provides combination therapies in which the amounts of each therapeutic may or may not be, on their own, therapeutically effective. For example, provided are methods comprising administering a first therapy and a second therapy in amounts that together are effective to treat or ameliorate a disorder, e.g., cancer. In some embodiments, at least one of the first and second therapies is administered to the subject in a lower effective dose. In some embodiments, both the first and the second therapies are administered in lower effective doses.

In some embodiments, the first therapy comprises a radiopharmaceutical and the second therapy comprises a checkpoint inhibitor.

In some embodiments, the first therapy comprises a checkpoint inhibitor and the second therapy comprises a radiopharmaceutical.

In some embodiments, therapeutic combinations as disclosed herein are administered to a subject in a manner (e.g., dosing amount and timing) sufficient to cure or at least partially arrest the symptoms of the disorder and its complications. In the context of a single therapy (a “monotherapy”), an amount adequate to accomplish this purpose is defined as a “therapeutically effective amount,” an amount of a compound sufficient to substantially improve at least one symptom associated with the disease or a medical condition. The “therapeutically effective amount” typically varies depending on the therapeutic. For known therapeutic agents, the relevant therapeutically effective amounts may be known to or readily determined by those of skill in the art.

For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. For example, a treatment may be therapeutically effective if it causes a cancer to regress or to slow the cancer's growth.

The dosage regimen (e.g., amounts of each therapeutic, relative timing of therapies, etc.) that is effective for these uses may depend on the severity of the disease or condition and the weight and general state of the subject. For example, the therapeutically effective amount of a particular composition comprising a therapeutic agent applied to mammals (e.g., humans) can be determined by the person of ordinary skill in the art with consideration of individual differences in age, weight, and the condition of the mammal. Because certain conjugates of the present disclosure exhibit an enhanced ability to target cancer cells and residualize, the dosage of these compounds can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agent. Therapeutically effective and/or optimal amounts can also be determined empirically by those of skill in the art. Thus, lower effective doses can also be determined by those of skill in the art.

Single or multiple administrations of a radiopharmaceutical or a composition (e.g., a pharmaceutical composition comprising a therapeutic agent or a radiopharmaceutical) can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

In the disclosed combination therapy methods, the first and second therapies may be administered sequentially or concurrently to a subject. For example, a first composition comprising a first therapeutic agent and a second composition comprising a second therapeutic agent may be administered sequentially or concurrently to a subject. Alternatively, a composition comprising a combination of a first therapeutic agent and a second therapeutic agent may be administered to the subject.

In some embodiments, the radiopharmaceutical is administered in a single dose. In some embodiments, the radiopharmaceutical is administered more than once, i.e., multiple doses. When the radiopharmaceutical is administered more than once, the dose of each administration may be the same or different.

In some embodiments, the checkpoint inhibitor is administered in a single dose. In some embodiments, the checkpoint inhibitor is administered more than once, e.g., at least twice, at least three times, etc. In some embodiments, the checkpoint inhibitor is administered multiple times according to a regular or semi-regular schedule, e.g., once every approximately two weeks, once a week, twice a week, three times a week, or more than three times a week. When the checkpoint inhibitor is administered more than once, the dose of each administration may be the same or different. For example, the checkpoint inhibitor may be administered in an initial dose amount, and then subsequent dosages of the checkpoint inhibitor may be higher or lower than the initial dose amount.

In some embodiments, the first dose of the checkpoint inhibitor is administered at the same time as the first dose of the radiopharmaceutical. In some embodiments, the first dose of the checkpoint inhibitor is administered before the first dose of radiopharmaceutical. In some embodiments, the first dose of the checkpoint inhibitor is administered after the first dose of radiopharmaceutical. In some embodiments, subsequent doses of the checkpoint inhibitor are administered.

In some embodiments, the present disclosure provides methods comprising administering to a mammal an ²²⁵Ac-radiopharmaceutical at a dosage of less than 2 MBq/kg (e.g., less than 1 MBq/kg, less than 750 kBq/kg, less than 500 kBq/kg, less than 400 kBq/kg, less than 300 kBq/kg, less than 250 kBq/kg, less than 200 kBq/kg, less than 150 kBq/kg, less than 100 kBq/kg, or less than 50 kBq/kg) of body weight of said mammal. Each of the dose may be administered multiple times to the mammal.

In certain embodiments, said ²²⁵Ac-radiopharmaceutical can be administered at a dosage of between 2 MBq/kg and 1.5 MBq/kg, between 1.5 MBq/kg and 1 MBq/kg, between 1 MBq/kg and 900 kBq/kg, between 900 kBq/kg and 800 kBq/kg, between 800 kBq/kg and 700 kBq/kg, between 700 kBq/kg and 600 kBq/kg, between 600 kBq/kg and 500 kBq/kg, between 500 kBq/kg and 400 kBq/kg, between 400 kBq/kg and 300 kBq/kg, between 300 kBq/kg and 200 kBq/kg, between 200 kBq/kg and 100 kBq/kg, or between 100 kBq/kg and 50 kBq/kg. Each of the dose may be administered multiple times to the mammal.

In certain embodiments, said ²²⁵Ac-radiopharmaceutical can be administered at a dosage of about 2 MBq/kg, about 1.9 MBq/kg, about 1.8 MBq/kg, about 1.7 MBq/kg, about 1.6 MBq/kg, about 1.5 MBq/kg, about 1.4 MBq/kg, about 1.3 MBq/kg, about 1.2 MBq/kg, about 1.1 MBq/kg, about 1 MBq/kg, about 0.9 MBq/kg, about 0.8 MBq/kg, about 0.7MBq/kg, about 0.6 MBq/kg, about 0.5 MBq/kg, about 0.4 MBq/kg, about 0.3 MBq/kg, about 0.2 MBq/kg, about 0.1 MBq/kg, or about 0.05 MBq/kg. Each of the dose may be administered multiple times to the mammal.

In some embodiments, said ²²⁵Ac-radiopharmaceutical is administered at a dosage of less than 250 kBq/kg (e.g., about 240 kBq/kg, about 220 kBq/kg, about 200 kBq/kg, about 180 kBq/kg, about 160 kBq/kg, about 150 kBq/kg, about 140 kBq/kg, about 130 kBq/kg, about 120 kBq/kg, about 110 kBq/kg, or about 100 kBq/kg) of body weight of said mammal. Each of the dose may be administered multiple times to the mammal.

In some embodiments, said ²²⁵Ac-radiopharmaceutical is administered at a dosage of less than 100 kBq/kg (e.g., about 90 kBq/kg, about 80 kBq/kg, about 70 kBq/kg, about 60 kBq/kg, about 50 kBq/kg, about 40 kBq/kg, about 30 kBq/kg, about 20 kBq/kg, or about 10 kBq/kg) of body weight of said mammal. Each of the dose may be administered multiple times to the mammal.

In some embodiments, said ²²⁵Ac-radiopharmaceutical is administered as a unitary dosage of less than 15 MBq (e.g., about 14 MBq, about 13 MBq, about 12 MBq, about 11 MBq, about 10 MBq, about 9 MBq, about 8 MBq, about 7 MBq, about 6 MBq, about 5 MBq, about 4 MBq, about 3 MBq, about 2 MBq, about 1 MBq) to said mammal. Each of the unitary dosage may be administered multiple times to the mammal.

In some embodiments, said ²²⁵Ac-radiopharmaceutical is administered as a unitary dosage of less than 10 MBq to said mammal. Each of the unitary dosage may be administered multiple times to the mammal.

In some embodiments, said ²²⁵Ac-radiopharmaceutical is administered as a unitary dosage of less than 5 MBq to said mammal. Each of the unitary dosage may be administered multiple times to the mammal.

In some embodiments, radiopharmaceuticals (or a composition thereof) and checkpoint inhibitors (or a composition thereof) are administered within 28 days (e.g., within 14, 7, 6, 5, 4, 3, 2, or 1 day(s)) of each other.

In some embodiments, radiopharmaceuticals (or a composition thereof) and checkpoint inhibitors (or a composition thereof) are administered within 90 days (e.g., within 80, 70, 60, 50, 40, 30, 20, 10, 5, 4, 3, 2, or 1 day(s)) of each other. In various embodiments the checkpoint inhibitor is administered at the same time as radiopharmaceutical. In various embodiments, the checkpoint inhibitor is administered multiple times after the first administration of radiopharmaceutical.

In some embodiments, compositions (such as compositions comprising radiopharmaceuticals) are administered for radiation treatment planning or diagnostic purposes. When administered for radiation treatment planning or diagnostic purposes, compositions may be administered to a subject in a diagnostically effective dose and/or an amount effective to determine the therapeutically effective dose. In some embodiments, a first dose of disclosed conjugate or a composition (e.g., pharmaceutical composition) thereof is administered in an amount effective for radiation treatment planning, followed administration of a combination therapy including a conjugate as disclosed herein and another therapeutic.

Pharmaceutical compositions comprising one or more agents (e.g., radiopharmaceuticals and/or checkpoint inhibitors) can be formulated for use in accordance with disclosed methods and systems in a variety of drug delivery systems. One or more physiologically acceptable excipients or carriers can also be included in the composition for proper formulation. Examples of suitable formulations are found in Remington's Pharmaceutical Sciences, Mack Publishing Company, Philadelphia, PA, 17th ed., 1985. For a brief review of methods for drug delivery, see, e.g., Langer (Science 249:1527-1533, 1990).

### Formulations

Pharmaceutical compositions may be formulated for parenteral, intranasal, topical, oral, or local administration, such as by a transdermal means, for prophylactic and/or therapeutic treatment. Pharmaceutical compositions can be administered parenterally (e.g., by intravenous, intramuscular, or subcutaneous injection), or by oral ingestion, or by topical application or intraarticular injection at areas affected by the vascular or cancer condition. Examples of additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Also specifically contemplated are sustained release administration, by such means as depot injections or erodible implants or components. Suitable compositions include compositions comprising include agents (e.g., compounds as disclosed herein) dissolved or suspended in an acceptable carrier, preferably an aqueous carrier, e.g., water, buffered water, saline, or PBS, among others, e.g., for parenteral administration. Compositions may contain pharmaceutically acceptable auxiliary substances to approximate physiological conditions, such as pH adjusting and buffering agents, tonicity adjusting agents, wetting agents, or detergents, among others. In some embodiments, compositions are formulated for oral delivery; for example, compositions may contain inert ingredients such as binders or fillers for the formulation of a unit dosage form, such as a tablet or a capsule. In some embodiments, compositions are formulated for local administration; for example, compositions may contain inert ingredients such as solvents or emulsifiers for the formulation of a cream, an ointment, a gel, a paste, or an eye drop.

Compositions may be sterilized, e.g., by conventional sterilization techniques, or sterile filtered. Aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 6 and 7, such as 6 to 6.5. In some embodiments, compositions in solid form are packaged in multiple single dose units, each containing a fixed amount of the above-mentioned agent or agents, such as in a sealed package of tablets or capsules. In some embodiments, compositions in solid form are packaged in a container for a flexible quantity, such as in a squeezable tube designed for a topically applicable cream or ointment.

### Effects

In some embodiments, methods of the present disclosure result in a therapeutic effect

In some embodiments, the therapeutic effect comprises a decrease in tumor volume, a stable tumor volume, or a reduced rate of increase in tumor volume. In some embodiments, the therapeutic effect comprises a decreased incidence of recurrence or metastasis.

### Other Agents

In some embodiments, disclosed methods further include administering an antiproliferative agent, radiation sensitizer, or an immunoregulatory or immunomodulatory agent.

By “antiproliferative” or “antiproliferative agent,” as used interchangeably herein, is meant any anticancer agent, including those antiproliferative agents listed in Table 1, any of which can be used in combination with a radiopharmaceutical to treat a condition or disorder. Antiproliferative agents also include organo-platinum derivatives, naphtoquinone and benzoquinone derivatives, chrysophanic acid and anthroquinone derivatives thereof.

By “immunoregulatory agent” or “immunomodulatory agent,” as used interchangeably herein, is meant any immuno-modulator, including those listed in Table 1, any of which can be used in combination with a radiopharmaceutical provided herein.

As used herein, “radiation sensitizer” includes any agent that increases the sensitivity of cancer cells to radiation therapy. Radiation sensitizers may include, but are not limited to, 5-fluorouracil, analogs of platinum (e.g., cisplatin, carboplatin, oxaliplatin), gemcitabine, EGFR antagonists (e.g., cetuximab, gefitinib), farnesyltransferase inhibitors, COX-2 inhibitors, bFGF antagonists, and VEGF antagonists.

TABLE 1

| | | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | Semustine |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | Miriplatin |
| | picoplatin | AP-5280 (Access) |
| | oxaliplatin | cisplatin |
| | carboplatin | |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (TopoTarget) | elsamitrucin (Spectrum) |
| | pixantrone (Novuspharma) | Edotecarin |
| | rebeccamycin analogue (Exelixis) | Cositecan |
| | BBR-3576 (Novuspharma) | Belotecan |
| | rubitecan (SuperGen) | hydroxycamptothecin (SN-38) |
| | irinotecan (CPT-11) | |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | Sabarubicin |
| | porfiromycin | Epirubicin |
| | mitoxantrone (novantrone) | mitoxantrone |
| | amonafide | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B |
| | T 900607 (Tularik) | (PharmaMar) |
| | T 138067 (Tularik) | ZD 6126 (AstraZeneca) |
| | cryptophycin 52 (Eli Lilly) | AZ10992 (Asahi) |
| | vinflunine (Fabre) | IDN-5109 (Indena) |
| | auristatin PE (Teikoku Hormone) | AVLB (Prescient NeuroPharma) |
| | BMS 247550 (BMS) | azaepothilone B (BMS) |
| | BMS 184476 (BMS) | BNP-7787 (BioNumerik) |
| | BMS 188797 (BMS) | CA-4 prodrug (OXIGENE) |
| | taxoprexin (Protarga) | dolastatin-10 (NIH) |
| | SB 408075 (GlaxoSmithKline) | CA-4 (OXIGENE) |
| | Vinorelbine | docetaxel |
| | Trichostatin A | vincristine |
| | | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum |
| | thymectacin (NewBiotics) | Pharmaceuticals) |
| | | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli |
| | tariquidar (Xenova) | Lilly) |
| | MS-209 (Schering AG) | biricodar dicitrate (Vertex) |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno-modulators | interferon | dexosome therapy (Anosys) |
| | oncophage (Antigenics) | pentrix (Australian Cancer |
| | GMK (Progenics) | Technology) |
| | adenocarcinoma vaccine (Biomira) | ISF-154 (Tragen) |
| | CTP-37 (AVI BioPharma) | cancer vaccine (Intercell) |
| | IRX-2 (Immuno-Rx) | norelin (Biostar) |
| | PEP-005 (Peplin Biotech) | BLP-25 (Biomira) |
| | synchrovax vaccines (CTL Immuno) | MGV (Progenics) |
| | melanoma vaccine (CTL Immuno) | ß-alethine (Dovetail) |
| | p21 RAS vaccine (Gem Vax) | CLL therapy (Vasogen) |
| | MAGE-A3 (GSK) | Ipilimumab (BMS), |
| | nivolumab (BMS) | CM-10 (cCam Biotherapeutics) |
| | abatacept (BMS) | atezolizumab (Genentech) |
| | pembrolizumab (Merck) | |
| Hormonal and | estrogens | dexamethasone |
| antihormonal agents | conjugated estrogens | prednisone |
| | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; | P-04 (Novogen) |
| | fluoxymesterone | 2-methoxyestradiol (EntreMed) |
| | methyltestosterone | arzoxifene (Eli Lilly) |
| | diethylstilbestrol | tamoxifen |
| | megestrol | toremofine |
| | bicalutamide | goserelin |
| | flutamide | Leuporelin |
| | nilutamide | bicalutamide |
| Photodynamic | talaporfin (Light Sciences) | Pd-bacteriopheophorbide (Yeda) |
| agents | Theralux (Theratechnologies) | Motexafin lutetium |
| | motexafin gadolinium | hypericin |
| | (Pharmacyclics) | |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | OSI-774 (Tarceva ™) | Gefitinib (Iressa) |
| | CI-1033 (Pfizer) | PTK787 (Novartis) |
| | SU11248 (Pharmacia) | EMD 72000 (Merck) |
| | RH3 (York Medical) | Emodin |
| | Genistein | Radicinol |
| | Radicinol | Vemurafenib (B-Raf enzyme |
| | Met-MAb (Roche) | inhibitor, Daiichi Sankyo) |
| | SR-27897 (CCK A inhibitor, Sanofi-Synthelabo) | ceflatonin (apoptosis promotor, ChemGenex) |
| | tocladesine (cyclic AMP agonist, Ribapharm) | BCX-1777 (PNP inhibitor, BioCryst) |
| | alvocidib (CDK inhibitor, Aventis) | ranpirnase (ribonuclease stimulant, Alfacell) |
| | CV-247 (COX-2 inhibitor, Ivy Medical) | galarubicin (RNA synthesis inhibitor, Dong-A) |
| | P54 (COX-2 inhibitor, Phytopharm) | tirapazamine (reducing agent, SRI |
| | CapCell ™ (CYP450 stimulant, Bavarian Nordic) | International) |
| | GCS-100 (gal3 antagonist, GlycoGenesys) | N-acetylcysteine (reducing agent, Zambon) |
| | G17DT immunogen (gastrin inhibitor, Aphton) | R-flurbiprofen (NF-kappaB inhibitor, Encore) |
| | efaproxiral (oxygenator, Allos Therapeutics) | 3CPA (NF-kappaB inhibitor, Active Biotech) |
| | PI-88 (heparanase inhibitor, Progen) | seocalcitol (vitamin D receptor agonist, Leo) |
| | tesmilifene (histamine antagonist, YM | 131-I-TM-601 (DNA antagonist, |
| | BioSciences) | TransMolecular) |
| | histamine (histamine H2 receptor agonist, | eflornithine (ODC inhibitor, ILEX Oncology) |
| | Maxim) | minodronic acid (osteoclast inhibitor, |
| | tiazofurin (IMPDH inhibitor, Ribapharm) | Yamanouchi) |
| | cilengitide (integrin antagonist, Merck KGaA) | indisulam (p53 stimulant, Eisai) |
| | SR-31747 (IL-1 antagonist, Sanofi-Synthelabo) | aplidine (PPT inhibitor, PharmaMar) |
| | CCI-779 (mTOR kinase inhibitor, Wyeth) | gemtuzumab (CD33 antibody, Wyeth Ayerst) |
| | exisulind (PDE V inhibitor, Cell Pathways) | PG2 (hematopoiesis enhancer, Pharmagenesis) |
| | CP-461 (PDE V inhibitor, Cell Pathways) | Immunol ™ (triclosan oral rinse, Endo) |
| | AG-2037 (GARFT inhibitor, Pfizer) | triacetyluridine (uridine prodrug, Wellstat) |
| | WX-UK1 (plasminogen activator inhibitor, | SN-4071 (sarcoma agent, Signature |
| | Wilex) | BioScience) |
| | PBI-1402 (PMN stimulant, ProMetic | TransMID-107 ™ (immunotoxin, KS |
| | LifeSciences) | Biomedix) |
| | bortezomib (proteasome inhibitor, Millennium) | PCK-3145 (apoptosis promotor, |
| | SRL-172 (T cell stimulant, SR Pharma) | Procyon) |
| | TLK-286 (glutathione S transferase inhibitor, | doranidazole (apoptosis promotor, Pola) |
| | Telik) | CHS-828 (cytotoxic agent, Leo) |
| | PT-100 (growth factor agonist, Point | trans-retinoic acid (differentiator, NIH) |
| | Therapeutics) | MX6 (apoptosis promotor, MAXIA) |
| | midostaurin (PKC inhibitor, Novartis) | apomine (apoptosis promotor, ILEX |
| | bryostatin-1 (PKC stimulant, GPC Biotech) | Oncology) |
| | CDA-II (apoptosis promotor, Everlife) | urocidin (apoptosis promotor, Bioniche) |
| | SDX-101 (apoptosis promotor, Salmedix) | Ro-31-7453 (apoptosis promotor, La Roche) |
| | rituximab (CD20 antibody, Genentech | brostallicin (apoptosis promotor, Pharmacia) |
| | carmustine | β-lapachone |
| | Mitoxantrone | gelonin |
| | Bleomycin | cafestol |
| | Absinthin | kahweol |
| | Chrysophanic acid | caffeic acid |
| | Cesium oxides | Tyrphostin AG |
| | BRAF inhibitors, | PD-1 inhibitors |
| | PD-L1 inhibitors | CTLA-4 inhibitors |
| | MEK inhibitors | sorafenib |
| | bevacizumab | |
| | angiogenesis inhibitors | |
| | dabrafenib | |

### EXAMPLES

### Example 1. Synthesis of Radiopharmaceuticals Comprising Compound of Formula I

Compounds of Formula I, including stereoisomers thereof, are small molecule antagonists targeting PSMA, which can be radiolabeled with a radionuclide such as Lutetium-177 (¹⁷⁷Lu) or Actinium-225 (²²⁵Ac) to form radionuclide-chelated radiopharmaceuticals. The synthesis of the compound of Formula I (or its stereoisomers), or corresponding radionuclide-chelated radiopharmaceuticals, can be referred to the following documents: Weineisen M, et al. EJNMMI Research, 2014, 4:63; Weineisen M, et al. J Nucl Med 2015, 56:1169-1176; U.S. Pat. No. 11,129,912 B1; and WO 2018/108287 A1.

The following exemplary compound with the designated stereospecificity, i.e., Compound A, prepared according to the above documents was used in the in vivo studies provided in Examples 2-5 below.

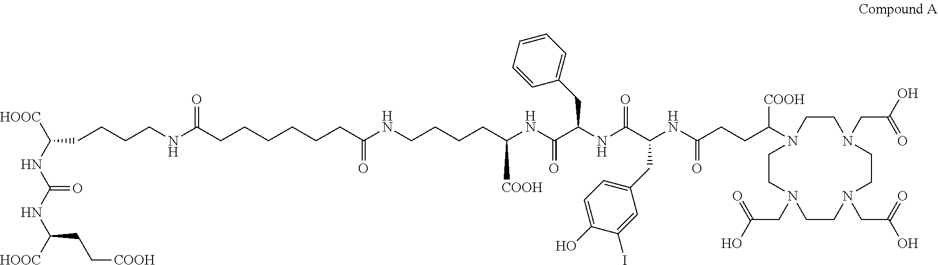 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

### Example 2. [¹⁷⁷Lu]-Compound A Biodistribution in the CT-26-mFOLH1 Syngeneic Immunocompetent Mouse Model

Compound A of Formula I was radiolabeled with Lu-177 using methods well known in the field to form [¹⁷⁷Lu]-Compound A. The ability of [¹⁷⁷Lu]-Compound A to target antigen expressing mouse PSMA overexpressing tumors in vivo was demonstrated using the CT-26-mFOLH1 syngeneic model. (The FOLH1 gene encodes PSMA.) Tumor uptake was maintained at 0.5-3% injected dose/g (ID/g) from 6-48 hours post injection. See FIG. 1.

### Example 3. Enhanced Efficacy of [²²⁵Ac]-Compound A in CT-26-mFOLH1 Syngeneic Immunocompetent Mouse Model

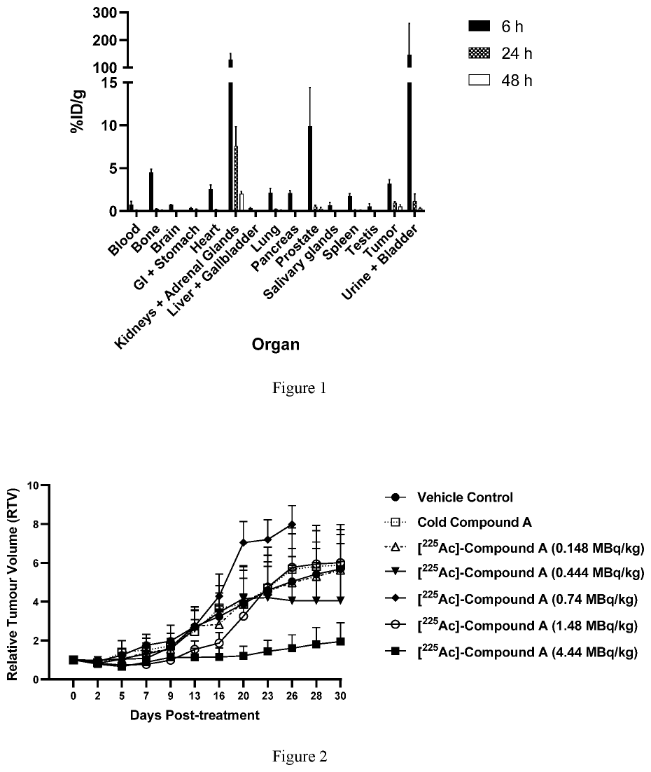

Compound A of Formula I was radiolabeled using standard techniques to form [²²⁵Ac]-Compound A. An efficacy study of [²²⁵Ac]-Compound A in immunocompetent mice was conducted using a 0.148 MBq/kg or 0.444 MBq/kg or 0.74 MBq/kg or 1.48 MBq/kg or 4.44 MBq/kg dose (single-dose, intravenous) of [²²⁵Ac]-Compound A. It was found that [²²⁵Ac]-Compound A at the highest dose tested (4.44 MBq/kg) had enhanced efficacy (as compared to cold compound A) in reducing tumor volume in CT-26-mFOLH1 Syngeneic mice with an intact immune system. See FIG. 2.

### Example 4. Combination of [²²⁵Ac]-Compound A and α-CTLA-4/PD-1 Treatment Resulted in Improved Efficacy in the CT-26-mFOLH1 Syngeneic Mouse Model

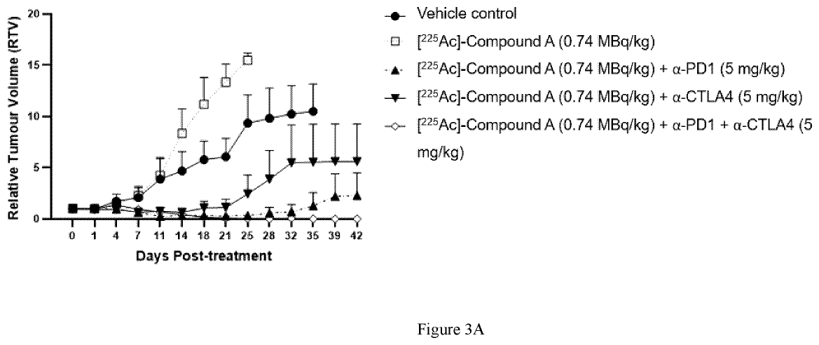

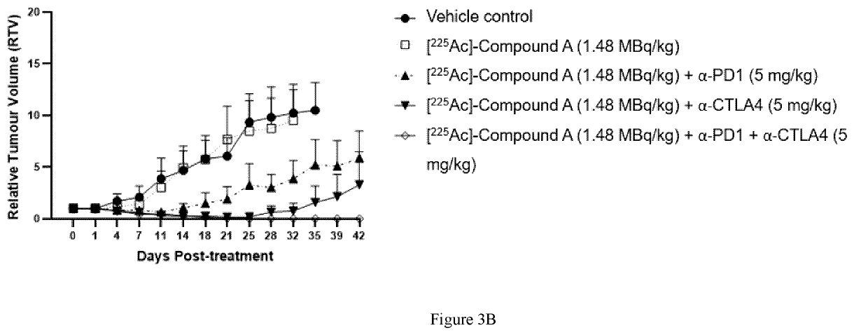

An in vivo study was conducted to test the effect of [²²⁵Ac]-Compound A (as described in Example 3) combined with checkpoint inhibitors, α-CTLA-4 and α-PD-1 antibodies, on relative tumor volume in the CT-26-mFOLH1 mouse model. When [²²⁵Ac]-Compound A was co-administered at a 0.74 MBq/kg dose (single-dose, intravenous) with either 5 mg/kg of α-CTLA-4 or α-PD-1, improved therapeutic efficacy was observed including tumor suppression. Co-administration with both α-CTLA-4 and α-PD-1 resulted in tumor regression and a significantly smaller tumor volume when compared to treatment with [²²⁵Ac]-Compound A in the presence or absence of either α-CTLA-4 or α-PD-1. See FIG. 3A

Separately, an in vivo study was conducted to test the effect of [²²⁵Ac]-Compound A at a different dosage combined with checkpoint inhibitors, α-CTLA-4 and α-PD-1 antibodies, on relative tumor volume in the CT-26-mFOLH1 mouse model. When [²²⁵Ac]-Compound A was co-administered at a 1.48 MBq/kg dose (single-dose, intravenous) with either 5 mg/kg of α-CTLA-4 or α-PD-1 or both, improved therapeutic efficacy was observed including tumor suppression, whereas co-administration with both α-CTLA-4 and α-PD-1 resulted in tumor regression, and significantly smaller tumor volume when compared to treatment with the [²²⁵Ac]-Compound A in the presence or absence of either α-CTLA-4 or α-PD-1. See FIG. 3B.

### Example 5. Improvement of the Overall Survival in [²²⁵Ac]-Compound A treated Mice

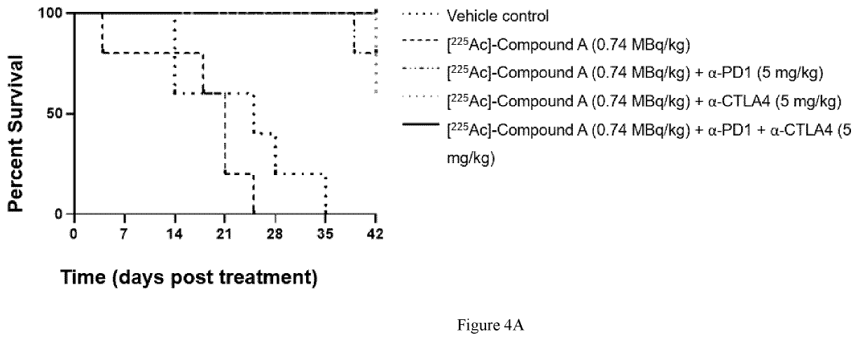

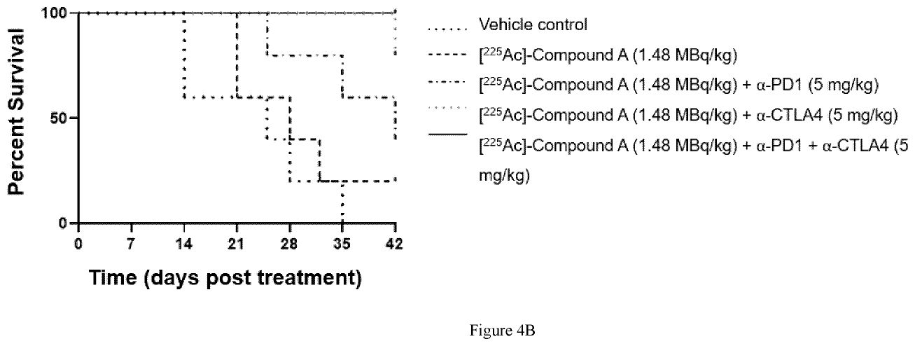

An in vivo study was conducted to test the effect of [²²⁵Ac]-Compound A (as described in Example 3) in combination with checkpoint inhibitors, α-CTLA-4 and α-PD-1 antibodies, on survival in the CT-26-mFOLH1 mouse model. When [²²⁵Ac]-Compound A was co-administered at a 0.74 MBq/kg dose with 5 mg/kg α-CTLA-4 or α-PD-1 (or both), the combination treatment resulted in improved overall survival compared to the vehicle control group or the monotherapy group ([²²⁵Ac]-Compound A alone). See FIG. 4A.

Separately, an in vivo study was conducted to test the effect of [²²⁵Ac]-Compound A at a different dosage combined with checkpoint inhibitors, α-CTLA-4 and α-PD-1 antibodies, on survival in the CT-26-mFOLH1 mouse model. When [²²⁵Ac]-Compound A was co-administered at a 1.48 MBq/kg dose with 5 mg/kg α-CTLA-4 or α-PD-1 (or both), the combination treatment resulted in improved overall survival. See FIG. 4B.

### Other Embodiments

Those skilled in the art will recognize or be able to ascertain using no more than routine experimentation, many equivalents to the specific embodiments described herein. Such equivalents are intended to be encompassed by the following claims.

1. A method for treating a mammal having cancers expressing Prostate Specific Membrane Antigen (PSMA), said method comprising:
(i) administering to the mammal an ²²⁵Ac-radiopharmaceutical, wherein the mammal has received or is receiving one or more checkpoint inhibitors;
(ii) administering to the mammal one or more checkpoint inhibitors, wherein the mammal has received or is receiving an ²²⁵Ac-radiopharmaceutical; or
(iii) administering to the mammal one or more checkpoint inhibitors at the same time as administering to the mammal an ²²⁵Ac-radiopharmaceutical,
wherein in each occurrence said ²²⁵Ac-radiopharmaceutical comprises ²²⁵Ac chelated with a compound of Formula I, or a stereoisomer thereof:

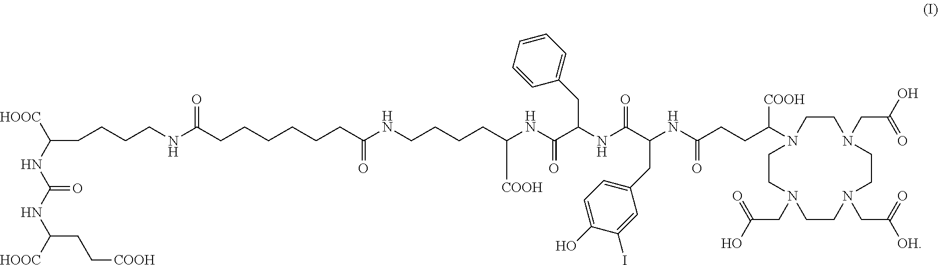 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

2. The method of claim 1, said method comprising administering to the mammal one or more checkpoint inhibitors, wherein the mammal has received or is receiving an ²²⁵Ac-radiopharmaceutical.

3. The method of claim 1 or 2, wherein said ²²⁵Ac-radiopharmaceutical comprises ²²⁵Ac chelated with the following structure:

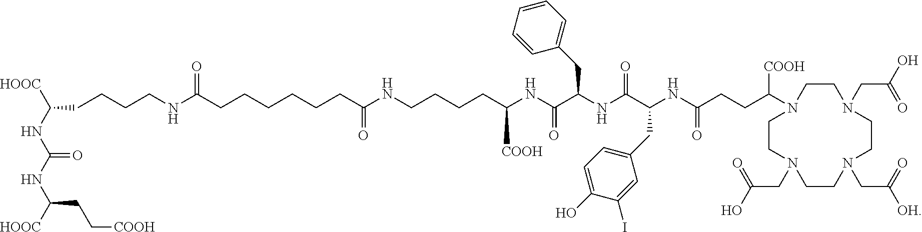 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

4. The method of any one of claims 1-3, wherein the one or more checkpoint inhibitors comprise a PD-1 or PD-L1 inhibitor or a CTLA-4 inhibitor.

5. The method of claim 4, wherein the PD-1 or PD-L1 inhibitor or the CTLA-4 inhibitor is an antibody.

6. The method of any one of claims 1-3, wherein the one or more checkpoint inhibitors comprise both a PD-1 or PD-L1 inhibitor and a CTLA-4 inhibitor.

7. The method of any one of claims 4-6, wherein the PD-1 or PD-L1 inhibitor is selected from the group consisting of camrelizumab, cemiplumab, dostarlimab, nivolumab, pembrolizumab, sintilimab, tislelizumab, toripalimab, RMP1-14, atezolizumab, avelumab, and durvalumab.

8. The method of any one of claims 4-6, wherein the CTLA-4 inhibitor is selected from the group consisting of BMS-986218, BMS-986249, ipilimumab, tremelimumab (formerly ticilimumab, CP-675,206), MK-1308, REGN-4659, and 4F10-11.

9. The method of any one of the preceding claims, wherein the mammal is a human.

10. The method of any one of the preceding claims, wherein said ²²⁵Ac-radiopharmaceutical is administered at a dosage of less than 2 MBq/kg of body weight of said mammal.

11. The method of any one of the preceding claims, wherein said ²²⁵Ac-radiopharmaceutical is administered at a dosage of less than 750 kBq/kg of body weight of said mammal.

12. The method of any one of the preceding claims, wherein said ²²⁵Ac-radiopharmaceutical is administered at a dosage of less than 250 kBq/kg of body weight of said mammal.

13. The method of any one of the preceding claims, wherein said ²²⁵Ac-radiopharmaceutical is administered as a unitary dosage of less than 15 MBq to said mammal.

14. The method of any one of the preceding claims, wherein said ²²⁵Ac-radiopharmaceutical is administered as a unitary dosage of less than 10 MBq to said mammal.

15. The method of any one of the preceding claims, wherein said ²²⁵Ac-radiopharmaceutical is administered as a unitary dosage of less than 5 MBq to said mammal.

16. The method of any one of the preceding claims, wherein the cancers are selected from the group consisting of prostate cancers, breast cancers, colorectal cancers, renal cell cancers, bladder cancers, testicular-embryonal cancers, neuroendocrine cancers, and brain tumors.

17. The method of claim 16, wherein the cancers are prostate cancers or breast cancers.

18. The method of any one of the preceding claims, wherein said administering results in a decrease in tumor volume, a stable tumor volume, or a reduced rate of increase in tumor volume.

19. The method of claim 18, wherein said administering results in a decreased incidence of recurrence or metastasis.

20. The method of claim 1, said method comprising administering to a mammal one or more checkpoint inhibitors, wherein the mammal has received or is receiving an ²²⁵Ac-radiopharmaceutical comprising ²²⁵Ac chelated with the following structure: 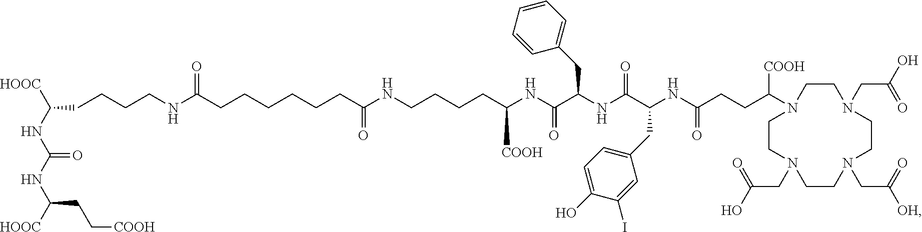 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]
wherein the one or more checkpoint inhibitors comprise both a PD-1 inhibitor and a CTLA-4 inhibitor, and wherein said ²²⁵Ac-radiopharmaceutical is administered at a dosage of 0.5-1.5 MBq/kg of body weight of said mammal.
